(19) United States
(12) Patent Application Publication
(10) Pub. No.: US 2026/0251665 A1
(43) Pub. Date: Aug. 27, 2026

(54) METHODS OF DETECTING A DISEASE OR CONDITION

(71) Applicant: BIOHABIT LTD, Cheshire (GB)

(72) Inventors: Vladislav Miscianinov, Manchester (GB); John David Hung, Liverpool (GB)

(21) Appl. No.: 18/882,000

(22) PCT Filed: Jul. 6, 2023
(86) PCT No.: PCT/GB2023/051785

§ 371 Date: Jan. 7, 2025

(30) Foreign Application Priority Data
Jul. 7, 2022 (GB) 2209983.2

(51) Int. Cl. G01N 33/74

(52) U.S. Cl. CPC G01N 33/74; G01N 2333/58; G01N 2800/325

(57) ABSTRACT

A method of aiding diagnosis of a disease or condition in a subject, wherein the method comprises comparing a level of at least one natriuretic peptide detected in a biological sample obtained from the subject with a reference level of the at least one natriuretic peptide, wherein the biological sample is an oral mucosal transudate (OMT) sample obtained from one or more mucous membranes inside the mouth of the subject.

### FIELD

The present invention relates to the detection and diagnosis of a disease or condition (such as a heart condition e.g. heart failure) in a subject, with the aid of a biomarker detectable in an oral mucosal transudate (OMT) sample obtained from the subject. The present invention also relates to methods of diagnosing the disease or condition (such as a heart condition e.g. heart failure) in a subject, and kits for use in diagnosing the disease or condition (such as a heart condition e.g. heart failure) in a subject.

### BACKGROUND

Heart failure remains a leading cause of global morbidity and mortality, associated with poor quality of life and significant healthcare costs. Mortality rates following diagnosis approach 43% at 5 years, with an economic burden of around $108 billion globally.

The clinical syndrome of heart failure results from an impairment in cardiac function and has multiple diverse aetiologies. Signs and symptoms are non-specific in the early stages of the disease, and clinical differentiation in primary care and non-emergent settings is challenging. Common symptoms such as dyspnoea, fatigue and leg swelling could be due to many other alternative diagnoses, and further tests are often required to make an ultimate diagnosis of heart failure, which may be expensive and time-consuming. This is particularly challenging where heart failure is in the early stages of the disease.

Serum biomarkers are known to be effective in detecting heart failure in a subject. For instance, natriuretic peptides including brain natriuretic peptide (BNP) and N-terminal prohormone B-type natriuretic peptide (NT-proBNP) are used as biomarkers to assist in the diagnosis of heart failure. During stretch of cardiac tissue (cardiac stretch) proBNP (prohormone B-type natriuretic peptide), a pro-hormone, is produced by the cardiac tissue e.g. cardiomyocytes. proBNP is then cleaved by furin, an endo-protease, giving rise to two fragments: (i) a biologically active 32-amino acid referred to as BNP, which corresponds to the C-terminal sequence of pro-BNP; and (ii) a 76-amino acid N-terminal fragment referred to as NT-proBNP. BNP and NT-proBNP are secreted from cardiac tissue e.g. cardiomyocytes and can be detected in blood. Biologically active BNP binds to natriuretic peptide receptors (NPR) on the kidneys and brain to exert its diuretic, vasodilatory and natriuretic effects.

Whilst both BNP and NT-proBNP blood tests are biomarkers for heart failure, due to better stability and longer half-life in vivo, NT-proBNP is considered to be the clinical gold standard in diagnosing heart failure. The half-life of NT-proBNP in blood is approximately 120 minutes, whereas the half-life of BNP in blood is approximately 20 minutes. In subjects with established heart failure, serum NT-proBNP is used as a biomarker to evaluate heart failure treatment efficacy and overall prognosis. If NT-proBNP levels in a subject's serum sample is greater than a reference level, it is indicative of heart failure in the subject. If NT-proBNP levels in a subject's serum sample are below the reference level, then it is considered safe to rule out heart failure in the subject.

There is a need for improved point-of-care diagnostics and evaluation of treatment in patients with heart failure. In particular, there is a need to develop point-of-care diagnostics which can rule out heart failure in the early stages of the disease.

It may be desirable to use bodily fluids other than blood for point of care diagnostics, such as oral fluids and urine. These fluids may be obtained from a subject more easily relative to blood as the presence of a medical practitioner is not required.

Oral fluids have been used in biomolecule monitoring for diagnostic purposes. However, it has been previously reported that there is no correlation between the concentration of NT-proBNP found in whole saliva and the concentration of NT-proBNP found in serum. As such, whole saliva is eminently unsuitable for use in methods of diagnosing or evaluating heart failure, as the concentration of NT-proBNP therein does not reflect that found in the blood.

Similarly, it has also been reported that urine is not a reliable biological sample for evaluating the NT-proBNP levels in a subject. One study suggests there is an inverse correlation between the concentration of NT-proBNP in urine and that found in serum, whereas another study suggests there is a positive correlation. These conflicting studies demonstrate that urine is eminently unsuitable for use in methods of diagnosing or evaluating heart failure because the correlation between urine NT-proBNP and serum NT-proBNP is reported to vary between populations selected for study.

There is, therefore, a need to find new and improved methods for diagnosing heart failure in a subject, and for evaluating heart failure in a subject.

It is an object of the present invention to mitigate or obviate one or more of the problems described above.

### SUMMARY OF INVENTION

The present disclosure concerns the use of OMT samples comprising a natriuretic peptide as a biomarker in the detection, prognosis and/or diagnosis of a disease or condition in a subject. More particularly, the present disclosure relates to the detection, prognosis and/or diagnosis of a heart condition in a subject. The present disclosure particularly concerns the use of OMT samples comprising N-terminal pro-brain natriuretic peptide (NT-proBNP) as a biomarker in the detection, prognosis and/or diagnosis of a heart condition in a subject, wherein the heart condition is heart failure.

The inventors have demonstrated, for the first time that NT-proBNP can surprisingly be detected in an OMT sample obtained from a subject. Moreover, the inventors have demonstrated that there is a surprising and unexpectedly strong correlation between the concentration of NT-proBNP in OMT, and the concentration of NT-proBNP in serum. In contrast, the inventors demonstrate that there is no correlation between the concentration of NT-proBNP in whole saliva and NT-proBNP in serum, in line with previous reports.

The inventors have further demonstrated that unexpectedly, OMT NT-proBNP provides high sensitivity and good specificity in demonstrating that OMT NT-proBNP is eminently suitable for methods of diagnosis and/or methods of evaluating diseases or conditions linked with elevated levels of NT-proBNP in a subject. The inventors have therefore demonstrated that OMT and detection of NT-proBNP therein is an effective and efficient alternative to detecting NT-proBNP in serum.

The inventors have thus surprisingly and unexpectedly discovered that OMT is particularly suitable for diagnosis of heart failure in a subject.

The inventors have provided a point of care diagnostic test utilising OMT, which is rapid, non-invasive, and cost-efficient relative to the current serum standard, to provide faster diagnosis and a reduction in healthcare costs. Such a point of care diagnostic method has particular advantages when applied in primary care (e.g. by a general practitioner), and in remote or rural environments where professional medical assistance and equipment is unavailable.

The present invention may advantageously be used to rule out heart failure in a subject at an early stage of diagnostic investigation, thus obviating the need for time-consuming and expensive cardiac tests and reducing the burden on the medical practitioner. The present invention may advantageously reduce the time it takes to refer a patient to a cardiologist, thus improving patient prognosis. As the present invention requires a sample that is easily obtained from a patient, the present invention may advantageously be used in heart condition screening, thus identifying patients which have the heart condition before they present as a medical emergency. The patient can then quickly be referred to a medical practitioner for further investigations, thus reducing the pressure on emergency doctors and improving patient prognosis. In short, the present invention provides rapid results, efficiently rules out heart failure in a subject, reduces costs, and reduces human effort relative to the clinical standard of serum analysis.

According to the present invention, there is provided a method of aiding diagnosis of a disease or condition in a subject, wherein the method comprises comparing a level of at least one natriuretic peptide detected in a biological sample obtained from the subject with a reference level of the at least one natriuretic peptide, wherein the biological sample is an OMT sample obtained from one or more mucous membranes inside the mouth of the subject. Preferably, the disease or condition is a heart condition. Preferably, the heart condition is heart failure.

According to present invention, there is provided a method of aiding diagnosis of a heart condition in a subject, wherein the method comprises comparing a level of at least one natriuretic peptide detected in a biological sample obtained from the subject with a reference level of the at least one natriuretic peptide, wherein the biological sample is an OMT sample obtained from one or more mucous membranes inside the mouth of the subject.

The one or more mucous membranes inside the mouth of the subject may be located at the gingiva, buccal mucosa, alveolar mucosa, labial mucosa, junctional epithelium, or combinations thereof.

The biological sample may be an OMT sample obtained from the surface of the one or more mucous membranes inside the mouth of the subject. The OMT sample obtained from the subject may be obtained from the surface of the gingiva in the mouth of the subject. The OMT sample may be obtained from the surfaces of the gingiva and the alveolar mucosa in the mouth of the subject. The OMT sample may be obtained from the surfaces of the gingiva, alveolar mucosa and labial mucosa in the mouth of the subject. The OMT sample may be obtained from the surfaces of the gingiva, alveolar mucosa and buccal mucosa in the mouth of the subject.

The OMT sample may be obtained from a particular location of one or more mucous membranes inside the mouth of the subject. The OMT sample may be obtained from a particular location of the gingiva. The OMT sample may be obtained from a particular location of the gingiva where the particular location is one or more of the gingival sulcus, the marginal gingiva, free gingiva, interdental gingiva, gingival groove, mucogingival junction and the attached gingiva. The particular location may be from the gingival sulcus.

The OMT sample may comprise gingival crevicular fluid. The OMT sample may be gingival crevicular fluid.

The at least one natriuretic peptide may be selected from BNP and NT-proBNP. The at least one natriuretic peptide may be NT-proBNP.

The concentration of the at least one natriuretic peptide in the OMT sample obtained from the subject may be from 0.5 pg/mL to 200 pg/mL, optionally from 1 pg/mL to 50 pg/mL, optionally still from 1 pg/mL to 40 pg/mL.

The disease or condition is preferably a heart condition. The heart condition may be selected from myocardial infarction, myocarditis, acute myocarditis, systolic and diastolic dysfunction, tachycardia, right-ventricular disorders (e.g. right ventricular overload), left-ventricular disorders (e.g. left ventricular hypertrophy), chronic obstructive pulmonary disease (COPD), heart attack, heart failure or combinations thereof. The heart condition is preferably heart failure.

Other diseases or conditions may include ischaemia, sepsis, leukaemia (e.g. acute myeloid leukemia), hypoxaemia, kidney conditions (e.g. chronic kidney disease), liver conditions (e.g. cirrhosis of the liver), diabetes and neurological conditions (e.g. dementia). These conditions have been associated with elevated levels of natriuretic peptides in a subject, more particularly with elevated levels of NT-proBNP.

The method may comprise determining whether the level of the at least one natriuretic peptide in the biological sample is greater than the reference level. The level of the at least one natriuretic peptide detected in the biological sample may be indicative of the disease or condition (e.g. heart failure) when the level of the at least one natriuretic peptide detected in the biological sample is greater than the reference level of natriuretic peptide. The level of the at least one natriuretic peptide detected in the biological sample may be indicative of the disease or condition (e.g. heart failure) when the level of the at least one natriuretic peptide detected in the biological sample is increased compared to the reference level of natriuretic peptide, optionally by at least 1%, optionally by at least 5%, and optionally by at least 10%. The level of the at least one natriuretic peptide detected in the biological sample may be indicative of the disease or condition (e.g. heart failure) when the level of the at least one natriuretic peptide detected in the biological sample is statistically significantly increased compared to the reference level of the at least one natriuretic peptide.

The method may comprise determining whether the level of the at least one natriuretic peptide is less than the reference level. If the level of the at least one natriuretic peptide is less than the reference level, then it is considered safe to rule out the subject having the disease or condition (e.g. a heart condition such as heart failure).

The method may comprise determining whether the level of the at least one natriuretic peptide is equal to the reference level. Equal to may be for example an increase within 0.1%, optionally within 1% of the reference value. If the level of the at least one natriuretic peptide is equal to the reference level then further clinical investigations may be required to provide a diagnosis.

The method may comprise the step of detecting the level of the at least one natriuretic peptide in the biological sample obtained from the subject. Detecting the level of the at least one natriuretic peptide in the biological sample obtained from the subject may comprise determining the concentration of the at least one natriuretic peptide in the OMT sample; and determining whether the concentration of the at least one natriuretic peptide in the OMT sample is indicative of the presence of the disease or condition (e.g a heart condition such as heart failure) in the subject. The step of determining may include determining whether the concentration of the at least one natriuretic peptide in the OMT sample is greater than the reference level. The step of determining may include determining whether the concentration of the at least one natriuretic peptide in the OMT sample is less than the reference level.

The reference level may be a reference concentration of the at least one natriuretic peptide in an OMT sample. The reference level may be pre-determined. For example, the reference level may be pre-determined based on pre-existing clinical standards. The method may include the step of determining the reference level. The reference level may be determined by: (i) determining the concentration of the at least one natriuretic peptide in one or more OMT samples independently obtained from one or more reference subjects who do not have the disease or condition (e.g. heart condition such as heart failure) or who have not been identified as having the disease or condition (e.g. heart condition such as heart failure); (ii) ranking the concentrations of the at least one natriuretic peptide detected in the one or more OMT samples obtained from the one or more reference subjects; and (iii) selecting a suitable concentration as the reference level. The ranking may be from smallest to largest. The reference level selected may be the 90^{th} percentile. For example, the reference level selected may be the 95^{th} percentile, optionally the 97^{th} percentile, further optionally the 99^{th} percentile, and optionally still to 100^{th} percentile. The reference level selected may be the 99^{th} percentile.

The OMT reference level may be a reference concentration of 100 pg/mL or less, optionally 50 pg/mL or less, optionally still 20 pg/mL or less. The OMT reference level may be a reference concentration of 10 pg/mL or less, optionally 8 pg/mL or less, optionally 5 pg/mL or less, preferably 4 pg/mL or less. The volume of the biological sample that is an OMT sample obtained from one or more mucous membranes inside the mouth of the subject may have a volume of from at least 50 μL to 2 mL OMT, optionally from at least 100 μL to 1 mL of OMT, and optionally still from at least 100 μL to 400 μL.

Also provided in accordance with the present invention is a method of diagnosing a disease or condition (e.g. heart condition such as heart failure) in a subject, wherein the method comprises comparing a level of at least one natriuretic peptide detected in a biological sample obtained from the subject with a reference level of the at least one natriuretic peptide, wherein the biological sample is an OMT sample obtained from one or more mucous membranes inside the mouth of the subject.

Also provided in accordance with the present invention is a method of aiding the assessment of a disease or condition (e.g. heart condition such as heart failure) in a subject identified as having or suspected of having the disease or condition (e.g a heart condition such as heart failure), comprises comparing the level of at least one natriuretic peptide detected in a first biological sample obtained from the subject before treatment for the disease or condition (e.g. heart condition), with the level of at least one natriuretic peptide detected in a second biological sample obtained from the subject during or after treatment for the disease or condition (e.g. heart condition), wherein the first and second biological samples are independently OMT samples obtained from one or more mucous membranes inside the mouth of the subject. The method may comprise detecting the level of the at least one natriuretic peptide in the first biological sample obtained from the subject before treatment for the disease or condition (e.g. heart condition); and detecting the level of the at least one natriuretic peptide in the second biological sample obtained from the subject during treatment for the disease or condition or after treatment for the disease or condition has ended (e.g. heart condition treatment). An increase in, or maintenance of, the level of the at least one natriuretic peptide from the first biological sample to the second biological sample may be indicative of an ineffective treatment for the disease or condition (e.g. heart condition treatment). A decrease in the level of the at least one natriuretic peptide from the first biological sample to the second biological sample may be indicative of an effective treatment for the disease or condition (e.g. heart condition treatment). If the levels of the at least one natriuretic peptide are equal then further clinical investigations may be required to provide a diagnosis.

The method may further comprise detecting a level of the at least one natriuretic peptide in at least one further biological sample obtained from the subject at a later time during or after the treatment for the disease or condition (e.g. heart condition treatment) than the second biological sample; and comparing the level of the at least one natriuretic peptide detected in the at least one further biological sample to the level of the at least one natriuretic peptide detected in the second biological sample. As such, the method may comprise for instance taking a third biological sample, and comparing the third with the second biological sample; then taking a fourth biological sample and comparing the fourth with the third biological sample, and so on. Each of the at least one further biological samples may be obtained independently from the subject during or after one or more independently administered treatments for the disease or condition (e.g. heart condition treatments). In this way, the level of the at least one natriuretic peptide in the biological sample obtained from the subject may be monitored routinely or continuously. Routine or continuous monitoring of levels of the one or more natriuretic peptides may advantageously provide useful prognostic information. For example, levels of the one or more natriuretic peptide could be regularly monitored to evaluate disease progression and response to treatment.

An increase in, or maintenance of, the level of the at least one natriuretic peptide from the second biological sample to the at least one further biological sample may be indicative of an ineffective treatment for the disease or condition (e.g. heart condition treatment). For example, an increase of at least 5%, or maintenance within 4% or less, of the level of the at least one natriuretic peptide between the biological samples being compared may be indicative of an ineffective treatment for the disease or condition (e.g. heart condition treatment).

A decrease in the level of the at least one natriuretic peptide from the second biological sample to the at least one further biological sample may be indicative of an effective treatment for the disease or condition (e.g. heart condition treatment). For example, a decrease of at least 5% of the level of the at least one natriuretic peptide between the biological samples being compared may be indicative of an effective treatment for the disease or condition (e.g. heart condition treatment). The reduction or increase in the level of the at least one natriuretic peptide between the biological samples being compared may be statistically significant.

Also provided in accordance with the invention is a method of assessing a disease or condition (e.g. a heart condition such as heart failure) in a subject identified as having or suspected of having the disease or disorder (e.g. a heart condition such as heart failure), comprising comparing the level of at least one natriuretic peptide detected in a first biological sample obtained from the subject before treatment for the disease or condition (e.g. treatment for the heart condition), with the level of at least one natriuretic peptide detected in a second biological sample obtained from the subject during or after treatment for the disease or condition (e.g. treatment for the heart condition), wherein the first and second biological samples are independently OMT samples obtained from one or more mucous membranes inside the mouth of the subject.

The level of the at least one natriuretic peptide in the biological sample may be determined using a mass spectrometry technique, an optical technique or a ligand-binding technique. The level of the at least one natriuretic peptide may be determined using a ligand-binding technique, wherein the ligand-binding technique is an immunogenic technique. The level of the at least one natriuretic peptide may be determined using an optical technique, such as using a spectrophotometer.

Also provided in accordance with the present invention is a kit for performing the methods according to the invention described herein, wherein the kit comprises one or more reagents for use in a method of detecting the at least one natriuretic peptide, optionally wherein the at least one natriuretic peptide is NT-proBNP.

Also provided in accordance with the present invention is OMT obtained from a subject for use in a method of diagnosing a disease or condition (e.g. a heart condition) in a subject, optionally wherein the disease or condition is a heart condition, optionally still wherein the heart condition is heart failure.

Also provided in accordance with the present invention is a method of aiding diagnosis of (or diagnosing) heart failure in a subject, wherein the method comprises comparing a level of NT-proBNP detected in a biological sample obtained from a subject with a reference level of NT-proBNP, wherein the biological sample is an OMT sample obtained from one or more mucous membranes inside the mouth of the subject selected from the gingiva, buccal mucosa, alveolar mucosa, labial mucosa, junctional epithelium, or combinations thereof. Optionally, the OMT sample is obtained from the gingiva. Optionally the OMT sample is obtained from the gingiva and one or more of the buccal mucosa, alveolar mucosa, labial mucosa, junctional epithelium.

Also provided in accordance with the present invention is a method of aiding the assessment of (or assessing) heart failure in a subject identified as having or suspected of having heart failure, the method comprising comparing the level of NT-proBNP detected in a first biological sample obtained from the subject before treatment for heart failure, with the level of NT-proBNP detected in a second biological sample obtained from the subject during or after treatment for heart failure, wherein the first and second biological samples are independently OMT samples obtained from one or more mucous membranes inside the mouth of the subject selected from the gingiva, buccal mucosa, alveolar mucosa, labial mucosa, junctional epithelium, or combinations thereof. Optionally, the OMT sample is obtained from the gingiva. Optionally the OMT sample is obtained from the gingiva and one or more of the buccal mucosa, alveolar mucosa, labial mucosa, junctional epithelium.

Also provided in accordance with the present invention is a method of ruling out a disease or condition (e.g. a heart condition such as heart failure) in a subject. Also provided in accordance with the present invention is a method of determining whether a subject is likely to have the disease or condition (e.g. a heart condition such as heart failure), the method being in accordance with the methods according to the invention described herein.

The methods described herein may be used as an indicator that the subject's condition requires further analysis to conclude whether the subject does have the disease or condition (e.g. a heart condition such as heart failure).

The methods of the present invention may further include the step of obtaining a biological sample from the subject. The step of obtaining a biological sample from the subject may include the steps of inserting a collection device into the mouth of the subject; and contacting the collection device with one or more mucous membranes inside the mouth for a period of time to collect the OMT sample. The collection device (e.g. an absorbent article) may collect at least 50 μL to 2 mL of OMT, optionally from 100 μL to 1 mL of OMT, and optionally still from 100-400 μL. If desired, two or more collection devices may be used, and optionally the samples that are independently collected in each collection device may be pooled to provide a single combined OMT sample.

Where the OMT sample is obtained from the surface of one or more mucosal membranes, the collection device may be an absorbent article which is brought into contact with the surface of one or more mucous membranes inside the mouth for a period of time to collect the OMT sample. The period of time may be at least 1 minute, optionally at least 3 minutes, optionally still at least 5 minutes. The period of time may be from 1 to 25 minutes, optionally from 3 to 20 minutes, optionally still from 5 to 15 minutes (e.g. from 5 to 10 minutes). Suitable absorbent articles include, for instance, oral swabs, sponges, and absorbent paper strips. For instance, collecting the OMT sample may include the steps of inserting the collection device (such as swab or sponge) into the mouth of the subject and positioning the collection device between the upper gum and cheek, and/or between the lower gum and cheek to contact the collection device with the one or more mucous membranes inside the mouth for a period of time.

Where the OMT sample is obtained from a particular location of one or more mucosal membranes, the collection device may be placed in contact with the particular location for a period of time. For instance, the collection device may be a needle under negative pressure located at the particular location of the one or more mucosal membranes from which the sample is to be collected. Particular locations may be, for instance, between the teeth and the gingiva, from the gingival sulcus, or from the floor of the mouth. For instance, where the OMT sample is gingival crevicular transudate collected from the gingival sulcus, it may be advantageous to use a collection device that is a needle under negative pressure to withdraw transudate from the desired location. Additionally or alternatively, the collection device may be paper strips which are located at a particular location of the one or more mucosal membranes from which the sample is to be collected (e.g. placed between a tooth and the gum at least partially in the gingival crevice). Additionally or alternatively, the collection device may be a micro-capillary located at the particular location of the one or more mucosal membranes from which the sample is to be collected.

The period of time may be at least 1 minute, optionally at least 3 minutes, optionally still at least 5 minutes. The period of time may be from 1 to 25 minutes, optionally from 3 to 20 minutes, optionally still from 5 to 15 minutes (e.g. from 5 to 10 minutes).

The OMT sample may be obtained from one or more mucous membranes in a first area of the inside of the mouth for a first period of time; and then from one or more mucous membranes in a second area of the inside of the mouth for a second period of time. The first and second periods of time may be the same, or different. Preferably, the first and second periods of time are the same.

The first area of the inside of the mouth may be the left hand side of the mouth, and the second area of the inside of the mouth may be the right hand side of the mouth. The first and second areas may be in areas of the same jaw (e.g. both areas of the upper jaw, or both areas of the lower jaw), or areas of different jaws (e.g. the upper jaw and the lower jaw). The first area of the inside of the mouth may be the left hand side of the lower jaw, and the second area of the inside of the mouth may be the right hand side of the lower jaw.

The first area and the second areas may contain the same one or more mucous membranes. For example, the OMT sample may be obtained from between the gingiva and the buccal mucosa on the left hand side of the lower jaw as the first area, and from between the gingiva and the buccal mucosa on the right hand side of the lower jaw as the second area. Equally, the OMT sample may be obtained from between the gingiva and the buccal mucosa on the left hand side of the lower jaw as the first area, and from between the gingiva and the buccal mucosa on the right hand side of the lower jaw as the second area.

The methods according to the present invention may find use in assessing or guiding a treatment plan for a patient with a disease or condition (e.g. a heart condition such as heart failure). For example, by detecting the level of a natriuretic peptide (e.g. NT-proBNP) in an OMT sample, before, during and/or after a treatment, it may be possible to ascertain if the treatment will, or is likely to, succeed based on how the level of the natriuretic peptide (e.g. NT-proBNP) in the oral fluid changes in response to the treatment. This assessment can also help to determine whether the treatment in question should be replaced with a different treatment. For example, treatments for heart failure can include, but are not limited to, medication, means to control a patient's heart rhythm, and surgery. Medication may be selected from ACE inhibitors, angiotensin-2 receptor blockers (ARBs), beta blockers, mineralocorticoid receptor antagonists, diuretics, ivabradine, sacubitril, valsartan, hydralazine with nitrate and digoxin. Means to control a patient's heart rhythm may be selected from pacemakers, cardiac resynchronisation therapy (CRT) devices, implantable cardioverter defibrillators (ICDs) and CRT-Ds. Surgery may be selected from heart valve surgery, a coronary angioplasty or bypass, left ventricular assist devices and heart transplant.

The methods and kits described herein may be used in aiding diagnosis of a disease or condition (e.g. a heart condition such as heart failure) at any stage and/or of any type of disease or condition (e.g. a heart condition such as heart failure). The methods and kits may be used for aiding diagnosis of a disease or condition (e.g. a heart condition such as heart failure).

Also described is a method of treating a subject identified as having or suspected of having a disease or condition (e.g. a heart condition such as heart failure), the method comprising the steps of diagnosing the subject as having the disease or condition by the methods of aiding diagnosis (or diagnosis) or methods of aiding the assessment of the disease or condition (or assessing) as described herein according to the present invention. Methods of treatment further comprise the step of administering a treatment to the subject for the disease or condition (e.g. a heart condition such as heart failure). The method of treatment may further comprise evaluating the efficacy of the treatment administered to the subject using the methods according to the present invention. The method of treatment may, if the subject is identified as having or suspected of having the disease or condition (e.g. a heart condition such as heart failure) after the initial treatment, further comprise the steps of administering the same or a different treatment to the subject for the disease or condition (e.g. heart condition such as heart failure).

The methods described herein may additionally be useful as methods of aiding prognosis of a disease or condition in a subject, or in methods of prognosis of a disease or condition in a subject. The subject may be identified as having or suspected of having a heart condition, such as heart failure. For instance, the methods described herein may be used to stratify patients or to triage patients identified as having or suspected of having a heart condition. For example, the methods described herein may be used to predict disease progression (e.g. progression of heart failure) or disease outcome (e.g. severity of disease, life expectancy, recovery). For example, OMT NT-proBNP may be used in a method of prognosis as described herein, wherein the disease or condition is heart failure.

As used herein, the term “aiding diagnosis” means at least assisting or guiding a diagnosis of a disease or condition such as a heart condition such as heart failure e.g. at least assisting or guiding a diagnosis of heart failure. In some instances, the term “aiding diagnosis” includes determining or confirming a diagnosis of a disease or condition such as a heart condition e.g. determining or confirming a diagnosis of heart failure. Thus, the methods described herein can be used in addition to existing methods of diagnosing the disease or condition such as a heart condition (e.g. heart failure), or may be used alone (i.e. without any existing methods of diagnosis) to diagnose the disease or condition such as a heart condition (e.g. heart failure).

As used herein, the term “diagnosis” refers to distinguishing between having and not having the disease or condition such as a heart condition e.g. between having and not having heart failure. As such, “diagnosis” may refer to distinguishing a subject as having, or at least likely to have, the disease or condition (such as a heart condition e.g. heart failure). As such, “diagnosis” may refer to distinguishing a subject as not having, or at least not likely to have, the disease or condition (such as a heart condition e.g. heart failure).

As used herein, the term “prognosis” refers to determining the likely course of a disease or condition in a subject e.g. recovery or progression of the disease or condition.

As used herein, the term “aiding assessment” means evaluating a disease or condition (such as a heart condition e.g. heart failure) in a subject where the subject is undergoing or has undergone treatment for the disease or condition (e.g. a heart condition such as heart failure).

The term “biological sample” as used herein is a sample obtained from the subject. Unless indicated otherwise, “biological sample” within the context of the present invention refers to an OMT sample obtained from one or more mucous membranes inside the mouth of the subject.

As used herein, the term “natriuretic peptide” refers to peptides which are hormones which induce natriuresis (excretion of sodium by the kidneys) and are produced by heart tissue. As used herein, the term “natriuretic peptide” further includes prohormones of said hormones, or fragments thereof. Within the context of the present invention “natriuretic peptides” include atrial natriuretic peptide (ANP), B-type natriuretic peptide (BNP), C-type natriuretic peptide (CNP), dendoroaspis natriuretic peptide (DNP) and urodilatin, prohormones thereof, and fragments thereof. Within the context of the present invention “natriuretic peptides” include fragments of the prohormone of BNP (proBNP), namely the hormone BNP and the biologically inactive fragment NT-proBNP.

As used herein, the term “BNP” refers to B-type (brain) natriuretic peptide. BNP is a 32-amino acid peptide which is a biologically active. BNP is the C-terminal end cleaved from prohormone BNP (pro-BNP).

As used herein, the term “NT-proBNP” refers to N-terminal prohormone of brain natriuretic peptide. NT-proBNP is a 76-amino acid biologically inactive protein which is the N-terminal end cleaved from prohormone BNP (pro-BNP).

As used herein, the term “serum NT-proBNP” refers to NT-proBNP comprised in a biological sample wherein the biological sample is serum obtained from a blood sample. As used herein, the term “whole saliva NT-proBNP” refers to NT-proBNP comprised in a biological sample wherein the biological sample is whole saliva. As used herein, the term “OMT NT-proBNP” refers to NT-proBNP comprised in a biological sample wherein the biological sample is OMT. As used herein, the term “urine NT-proBNP” refers to NT-proBNP comprised in a biological sample wherein the biological sample is urine.

As used herein, the term “serum” refers to a fraction of the blood from which clotting factors have been removed.

As used herein, the term “whole saliva” is the oral fluid secreted from parotid, submandibular, sublingual, and minor salivary glands. Whole saliva also contains traces of other oral fluids such as OMT and gingival crevicular fluid.

As used herein, the term “oral mucosal transudate” (OMT) refers to transudate from one or more mucous membranes inside the mouth of the subject. Put another way, OMT is secreted from one or more mucous membranes in the mouth. Due to close proximity of capillaries to the mucous membranes, OMT may also be considered a blood ultrafiltrate. For example, OMT may be considered to be a transudate from the capillaries beneath the buccal mucosa and at the base of the gingival sulcus. As such, OMT may comprise gingival crevicular fluid from the gingival sulcus. The composition of OMT is not well understood. Whilst OMT may be comprised in whole saliva by virtue of being an oral fluid, those skilled in the art understand that OMT is distinct from whole saliva as it is collected from specific regions of the oral cavity, namely one or more mucous membranes. Several biomolecules have been previously identified in OMT including antibodies and inflammatory cytokines. For instance, OMT is a clinically recognised sample that is used as a diagnostic fluid for HIV antibody testing.

As used herein, the term “oral mucosal transudate (OMT) sample” is not included in, or comprised in, whole saliva. The “OMT sample” may consist of OMT. Those skilled in the art will therefore appreciate that an OMT sample is distinct from a whole saliva sample. In other words, OMT is not whole saliva. Those skilled in the art will appreciate that the collection methods for OMT are distinct to collection methods for whole saliva. For instance, whole saliva may typically be collected by the ‘drool’ method, whereas OMT requires collection of transudate from one or more mucous membranes (e.g. using a swab). As described herein in more detail, the OMT sample may be obtained specifically from the surface of one or more mucous membranes inside the mouth of the subject. Alternatively or additionally, the OMT sample may be obtained from particular areas of one or more mucous membranes inside the mouth of the subject. For instance, gingival crevicular fluid (GCF) is obtained from particular areas of the gingiva.

As used herein, the term “gingival crevicular fluid” (GCF) refers an OMT derived from periodontal tissues containing serum components, inflammatory cytokines and chemokines and antibodies. “Gingival crevicular fluid” is also referred to as an inflammatory exudate. Periodontal tissues include the gingiva, cementum, alveolar bone, and the periodontal ligament. Gingival crevicular fluid is typically obtained from the gingiva. In particular, GCF is obtained from one or more of the junctional epithelium, marginal gingiva, free gingiva, the attached gingiva, interdental gingiva (papilla), alveolar mucosa, and the gingival sulcus (also referred to as the gingival crevice or gingival cavity). Typically, gingival crevicular fluid is obtained from the gingival sulcus. Those skilled in the art will appreciate that as OMT typically comprises GCF, then OMT may be obtained from the same areas from which GCF is generally obtained.

As used herein, the term “mucous membrane” refers to mucosa lining the inside of the mouth which secrete mucous. Mucous membranes inside the mouth include, but are not limited to, gingiva, buccal mucosa, alveolar mucosa, and labial mucosa. Mucous membranes may also be referred to as mucosal membranes.

As used herein, the term “gingiva” refers to the tissue that surrounds the base of the teeth of the upper and lower jaws. Gingiva may also be referred to as ‘gums’, and the extremities of the gingiva adjacent the teeth may be referred to as the ‘gum line’. Gingiva can include the marginal gingiva (gingiva proximal to the base of the teeth), interdental gingiva (also referred to as papilla), attached gingiva, free gingiva, junctional epithelium, and gingival sulcus (also referred to as the gingival crevice or gingival cavity). The junction between the gingiva and the alveolar mucosa is referred to as the mucogingival junction, and for the purposes of defining the invention, the mucogingival junction is considered to be included in the gingiva.

As used herein, the term “buccal mucosa” refers to the lining of the cheeks inside the mouth where they touch the teeth. Buccal mucosa may also refer to the back of the lips, inside the mouth where they touch the teeth. Buccal mucosa is located at lining of the cheeks proximal the upper jaw and the lower jaw.

As used herein, the term “alveolar mucosa” refers to the mucous membrane that sits above the gingiva of the upper jaw or below the gingiva of the lower jaw, and continues across the floor of the mouth, cheeks, and lips. As used herein, the “floor of the mouth” refers to the area underneath the tongue.

As used herein, the term “labial mucosa” refers to the lining of the inside of the lips. Labial mucosa is located at the lining of both the upper lip (of the upper jaw) and the lower lip (of the lower jaw).

As used herein, the term “surface” of one or more mucous membrane means a surface area of the one or more mucous membrane that is accessible in the oral cavity. For instance, the gingiva and buccal mucosa are accessible mucous membranes in the mouth.

The term “reference level” refers to a value (e.g. a concentration) against which other values are compared. The reference level may be any suitable value, but is typically based on known reference levels. The reference level may be a level of the at least one natriuretic peptide that is observed in one or more biological samples obtained from a healthy population (i.e. from subjects who do not have the disease or condition). For instance, reference levels may be set by medical agencies. For example the reference level for NT-proBNP in serum in Europe is 125 pg/mL, and in the UK the NT-proBNP serum reference level is 400 pg/mL. The serum NT-proBNP levels in selected other jurisdictions are as follows: Canada (125 pg/mL), Australia (300 pg/mL), New Zealand (300 pg/mL), USA (125 pg/mL for patients aged 0-74 years and 450 pg/mL for patients aged 75-99 years).

The terms “increased” and “reduced”, as used herein, refer to an upward or a downward deviation, respectively, in detected levels as compared to the reference level.

The term “detecting” or “detection”, as used herein, may be used in a qualitative or quantitative sense. Thus, the term “detecting” may simply refer to detecting a level in a qualitative manner and hence simply conveying whether or not the level of the at least one natriuretic peptide (e.g. NT-proBNP) is increased or reduced as compared to a reference level. Alternatively, “detection” may be understood to be more quantitative and may include determining a specific value or amount of the at least one natriuretic peptide (e.g. NT-proBNP) in the biological sample.

### BRIEF DESCRIPTION OF FIGURES

FIG. 1. NT-proBNP concentration range in healthy participants vs heart failure patients in whole saliva and OMT and their paired serum samples. NT-proBNP concentration is expressed in pg/mL. Data is plotted as median with range. *p<0.05; **p<0.01, ***p<0.001, ****p<0.0001 versus healthy. Mann-Whitney test is applied.

FIG. 1A: NT-proBNP concentrations of whole saliva samples in healthy participants (n=14) vs heart failure patients (n=21).

FIG. 1B: NT-proBNP concentrations of paired serum samples in healthy participants (n=14) vs heart failure patients (n=21).

FIG. 1C: NT-proBNP concentrations of OMT samples in healthy participants (n=7) vs heart failure patients (n=9).

FIG. 1D: NT-proBNP concentrations of paired serum samples in healthy participants (n=7) vs heart failure patients (n=9).

FIG. 2. NT-proBNP is detectable in whole saliva and does not correlate with serum NT-proBNP. Volume is expressed in μL; NT-proBNP concentration is expressed in pg/mL. Data is plotted as median with range.

FIG. 2A: Volume range of whole saliva samples. n=35.

FIG. 2B: NT-proBNP concentration range in whole saliva samples. n=35.

FIG. 2C: NT-proBNP concentration range in serum samples.

FIG. 2D: Correlation of whole saliva NT-proBNP concentrations vs serum NT-proBNP concentrations. Spearman's rank correlation coefficient is used due to non-normality of data. Spearman r=0.24, p=0.083; n=35.

FIG. 3. NT-proBNP is detectable in OMT and positively correlates with serum NT-proBNP. Volume is expressed in μL; NT-proBNP concentration is expressed in pg/mL. Data is plotted as median with range.

FIG. 3A: Volume range of OMT samples. n=16

FIG. 3B: NT-proBNP concentration range in OMT samples; n=16

FIG. 3C: NT-proBNP concentration range in serum samples; n=16

FIG. 3D: Correlation of OMT NT-proBNP concentrations vs serum NT-proBNP concentrations. Spearman's rank correlation coefficient is used due to non-normality of data. Spearman r=0.69, p=0.0021, p value summary is **; n=16

FIG. 4. OMT NT-proBNP ROC curve and ROC AUC analyses.

FIG. 4A: ROC analysis of healthy participants OMT NT-proBNP values vs heart failure OMT NT-proBNP. ROC curve data distributed as following: on the left side—OMT NT-proBNP values from healthy volunteers; on the right side—OMT NT-proBNP values from heart failure patients. Data is plotted as median with range.

FIG. 4B: ROC analysis of healthy participants OMT NT-proBNP values vs heart failure OMT NT-proBNP. ROC curve analysis expressed as % sensitivity vs % specificity of OMT NT-proBNP.

### DETAILED DESCRIPTION

The invention will now be described by way of example only, with reference to the accompanying Figures.

Detecting levels of NT-proBNP in an OMT sample obtained from a subject to aid the diagnosis of (or to diagnose) heart failure in a subject is described by way of example.

The detected level of OMT NT-proBNP is compared to a reference level of OMT NT-proBNP, and an increased level of OMT NT-proBNP compared to the reference level is indicative of heart failure. The reference level of OMT NT-proBNP, for comparison, may be determined from the level of NT-proBNP detected in one or more subjects who do not have heart failure, or who have not been identified as having heart failure.

### Subject

As used herein, a subject or a reference subject refers to a human subject. According to the present disclosure, a subject may present one or more clinical symptoms of the disease or condition e.g. heart failure. Clinical symptoms of heart failure will be known to those skilled in the art, but may include one or more of dyspnoea (breathlessness), fatigue, swollen ankles and legs, a fast heart rate, a pounding, fluttering or irregular heartbeat (palpitations).

The one or more reference subjects, as used herein, does not have the disease or condition (e.g. heart failure) or has not been identified as having the disease or condition (e.g. heart failure). Preferably, each of the one or more reference subjects does not present any clinical symptoms of the disease or condition (e.g. heart failure), as listed above.

### Biological Sample

Methods of obtaining biological samples, such as OMT, are well known in the art. Any known method may be used for obtaining the biological samples used in the methods of the present invention. Exemplary methods of obtaining biological samples are outlined in the examples below. Methods of obtaining blood are well known to those in the art. Methods of obtaining whole saliva may include the passive drooling method. Methods of obtaining OMT may include using the absorbency method (e.g. using a swab, absorbent collection pad, sponge or similar absorbent article). The methods described in accordance with the present invention may comprise a step of obtaining a biological sample from the subject.

Exemplary methods of obtaining OMT are described herein, but are known to those skilled in the art. Methods of obtaining OMT from the mouth of a subject may include contacting an absorbent article (e.g. a swab, sponge, or an absorbent pad) with one or more mucous membranes in the mouth of the user for a period of time. The absorbent article may be left stationary in the mouth of the user for a period of time at a desired location, or may be moved within the mouth of the user for a period of time. For instance, an absorbent pad may be gently dragged (swabbed) across the surface of the upper and lower gums of the subject.

### Detection of NT-proBNP

Detection of NT-proBNP may be carried out using any suitable technique, including ligand-binding techniques, such as immunogenic techniques using an antibody or antibody fragment specific for NT-proBNP or techniques using aptamers or molecularly imprinted polymers; mass spectrometry techniques; and optical techniques, such as microscopy techniques, imaging techniques, sensing techniques, and spectroscopy techniques. Spectroscopy techniques include Raman spectroscopy techniques, for example, Raman spectroscopy or surface enhanced Raman spectroscopy.

The ligand-binding technique may be an immunogenic technique. The immunogenic technique may be an immunoassay.

An antibody, such as a monoclonal antibody, may be used in a method as described herein. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996).

The term “immunoassay”, as used herein, encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); chemiluminescence assays (CL) and lateral/vertical flow assays. If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for NT-proBNP may be suitable for sensitive, non-radioactive detection of NT-proBNP. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

Detection of NT-proBNP may be carried out by mass spectrometry. The detection may be done by a mass spectrometer, which may be, for example, one of the following: MALDI-TOF MS, Tandem MS, ESI-TOF, ESI-iontrap, LC-MS, LC-MS/MS, GC-MS, ion mobility MS, laser desorption ionization mass spectrometry (LDI-MS) and quadrupole-MS. Other mass spectrometry devices and methods now existing or which may be developed in the future, are within the scope of the present invention.

Detection of NT-proBNP may be carried out by a Raman spectroscopy technique or surface enhanced Raman spectroscopy.

The methods described can be carried out in a variety of physical formats. For example, the use of micro titer plates or automation could be used to facilitate the processing of large numbers of test samples. Alternatively, single sample formats, including Eppendorf tubes, test tubes and the like may be used to facilitate diagnosis or prognosis in a timely fashion.

### Reference Level

The reference level of NT-proBNP may be threshold level of NT-proBNP in the biological sample, wherein if a measured level of NT-proBNP in a biological sample is found to be greater than the reference level, then this is considered to be indicative that the subject is likely to have (e.g. is suspected of having) heart failure, or may be indicative that the subject has heart failure. Equally, if the measured level of NT-proBNP in the biological sample is less than the reference level, then it is indicative that the subject is unlikely to have heart failure or does not have heart failure. If the measured level of NT-proBNP in the biological sample is equal to the reference level (e.g. within 1% of the reference level), then further clinical investigations may be required.

The reference level may be pre-determined. The method of aiding the diagnosis of a heart condition may include comparing the level of NT-proBNP detected in the biological sample obtained from the subject with a previously-determined reference level. In such instances, the method of aiding the diagnosis of heart failure may not require an additional step of determining the reference level of NT-proBNP, and the level of NT-proBNP detected in the subject in question can be compared directly to the previously-determined reference level.

The reference level of NT-proBNP may be a level of NT-proBNP derived from one or more biological samples obtained from one or more reference subjects who do not have heart failure or who have not been identified as having heart failure. The method of aiding the diagnosis of heart failure may comprise the steps of determining the concentrations of NT-proBNP in the biological samples obtained from one or more reference subjects who do not have heart failure or who have not been identified as having heart failure, ranking the concentrations, and selecting a suitable concentration as the reference level. The reference level selected is typically greater than the 90^{th} percentile. For example, the value selected may be the 95^{th} percentile, optionally the 97^{th} percentile, and optionally still the 99^{th} percentile.

Alternatively, the baseline level of NT-proBNP may be the average level of NT-proBNP detected in biological samples from one or more reference subjects who do not have heart failure or who have not been identified as having heart failure. The method of aiding the diagnosis of heart failure may comprise a step of detecting an average level of NT-proBNP in a biological sample obtained from each of one or more reference subjects who do not have heart failure or who have not been identified as having heart failure. This average level of NT-proBNP is used as the baseline level in the methods of the invention.

### Increased Level of NT-proBNP

A level of NT-proBNP detected in the biological sample of the subject is indicative of suspected heart failure, or of heart failure, when the detected level is greater than (increased relative to) the reference level of NT-proBNP. The greater the increase of NT-proBNP level in the biological sample relative to the reference level, the greater the likelihood is that the subject has heart failure. Patients with severe heart failure may have a level of NT-proBNP which is up to 500-fold greater than the reference level.

The detected level of NT-proBNP may be increased by at least 0.1%, optionally at least 5% compared to the reference level of NT-proBNP. The level of NT-proBNP detected in the biological sample of the subject may be indicative of heart failure if the detected level is increased by at least 5%, at least 10%, at least 15%, at least 20%, at least 25%, at least 30%, at least 35%, at least 40%, at least 45% or at least 50% compared to the reference level. An increase of at least 5%, optionally at least 10% in the biological sample obtained from the subject compared to the reference level may be indicative of heart failure.

An increase in the detected level of NT-proBNP from 0.1 to 2000% compared to the reference level of NT-proBNP, optionally from 10 to 1000%, optionally still from 20% to 500%, may be indicative of heart failure. For instance, an increase in the detected level of NT-proBNP from 40 to 400%, optionally from 50 to 300%, may be indicative of heart failure.

### Method of Assessing Heart Failure Treatment

In the method of aiding the assessment of heart failure treatment of a subject, the subject may have been previously identified as having heart failure. The method of the invention may include a step of identifying that a subject has heart failure, using existing techniques or the method of the present invention for aiding diagnosis, prognosis or grading of heart failure described above.

The method may comprise a step of administering a heart failure treatment to the subject after obtaining the first biological sample and before obtaining the second biological sample. The heart failure treatment can be any known heart failure treatment.

After obtaining the first biological sample from the subject and before obtaining the second biological sample, the subject may receive a heart failure treatment. The heart failure treatment can be any known heart failure treatment.

The first biological sample may be obtained 1 hour, 2 hours, 6 hours, 12 hours, 24 hours, 2 days, 3 days, 4 days, 5 days, 6 days, 7 days, 14 days or 1 month before the heart failure treatment.

The second biological sample may be obtained during heart failure treatment, and is obtained 1, 2, 3, 4, 5, 6, 7 or 8 weeks, or 2, 3, 4, 5, 6, 7, 8, 9, 10, 11, 12 or 13 months after heart failure treatment has started. The second biological sample may be obtained 1, 2, 3 or 4 months after heart failure treatment has started. The second biological sample may be obtained 1, 2, 3, 4, 5, 6, 7 or 8 weeks, or 2, 3, 4, 5, 6, 7, 8, 9, 10, 11 or 12 months after heart failure treatment has ended. The second biological sample may be obtained 1, 2, 3, 4, 2 weeks, or 2, 3 or 4 months, after heart failure treatment has ended.

The at least one further biological sample is obtained after the second biological sample. Thus, the at least one further biological sample may be obtained during heart failure treatment, and 1, 2, 3, 4, 5, 6, 7 or 8 weeks, or 2, 3, 4, 5, 6, 7, 8, 9, 10, 11, 12 or 13 months after heart failure treatment has started. The at least one further biological sample may be obtained 1, 2, 3, 4, 5, 6, or 7 weeks, or 2, 3 or 4 months after heart failure treatment has ended.

The second biological sample may be obtained during heart failure treatment and at least one further biological sample is obtained at a later time during heart failure treatment. The second biological sample may be obtained during heart failure treatment and at least one further biological sample is obtained after heart failure treatment has ended. The second biological sample may be obtained after heart failure treatment has ended and at least one further biological sample is obtained at a later time after heart failure treatment has ended.

An increase in the level of NT-proBNP detected in the second biological sample compared to the level detected in the first biological sample is indicative of the heart failure treatment being ineffective. Similarly, an increase in the level of NT-proBNP detected in the at least one further biological sample compared to the level detected in the second biological sample is indicative of the heart failure treatment being ineffective. The level of NT-proBNP detected in the second or at least one further biological sample is also indicative of the heart failure treatment being ineffective when it stays the same compared to the level detected in the first or second biological sample, respectively (i.e. when the level of NT-proBNP stays the same between the biological samples being compared).

An increase in the level of NT-proBNP between the biological samples being compared (i.e. from the first biological sample to the second biological sample, or from the second biological sample to the at least one further biological sample) may be indicative of ineffective heart failure treatment. The increase may be an increase of at least 5%, at least 10%, at least 15%, at least 20%, at least 25%, at least 30%, at least 35%, at least 40%, at least 45% or at least 50%. An increase in the level of NT-proBNP of at least 5%, optionally at least 10% between the biological samples being compared may be indicative of the heart failure treatment being ineffective. An increase in the detected level of NT-proBNP from 0.1 to 500% compared to the level of NT-proBNP in the comparison biological sample, optionally from 10 to 300%, optionally still from 15% to 200%, may be indicative of ineffective heart failure treatment. For instance, an increase in the detected level of NT-proBNP from 20 to 95%, optionally from 25 to 85%, may be indicative of may be indicative of ineffective heart failure treatment.

The level of NT-proBNP may be considered to have been maintained (stayed the same) between the biological samples being compared (i.e. from the first biological sample to the second biological sample, or from the second biological sample to the at least one further biological sample) when the change in the detected level of NT-proBNP is 1% or less, optionally 2% or less, optionally 4% or less, optionally 5% or less, optionally still 10% or less from the detected starting level of NT-proBNP in the comparison biological sample.

The method may comprise stopping the heart failure treatment being assessed when the comparison of the levels of NT-proBNP between the biological samples is indicative of the heart failure treatment being ineffective. Optionally, a different heart failure treatment can then be administered to the subject.

A decrease in the level of NT-proBNP detected in the second biological sample compared to the level detected in the first biological sample is indicative of the heart failure treatment being effective. Similarly, a decrease in the level of NT-proBNP detected in the at least one further biological sample compared to the level detected in the second biological sample is indicative of the heart failure treatment being effective.

A decrease in the level of NT-proBNP between the biological samples being compared (i.e. from the first biological sample to the second biological sample, or from the second biological sample to the at least one further biological sample) may be indicative of effective heart failure treatment when the decrease is of at least 5%, at least 10%, at least 15%, at least 20%, at least 25%, at least 30%, at least 35%, at least 40%, at least 45% or at least 50%. Preferably, a decrease in the level of NT-proBNP of at least 5%, optionally at least 10%, between the biological samples being compared is indicative of the heart failure treatment being effective. A decrease in the detected level of NT-proBNP from 5 to 95% compared to the reference level of NT-proBNP, optionally from 10 to 85%, optionally still from 15% to 75%, may be indicative of the heart failure treatment being effective. For instance, a decrease in the detected level of NT-proBNP from 20 to 65%, optionally from 25 to 55%, may be indicative of the heart failure treatment being effective.

### Type of Heart Failure

The methods of the invention are suitable for aiding the diagnosis of any type of heart failure, and for aiding the assessment of the treatment of any type of heart failure. Heart failure may be acute heart failure or decompensated heart failure, which can develop suddenly e.g. after myocardial infarction or as an acute complication of heart valve problems. Heart failure may be chronic heart failure, which is more common than acute heart failure with symptoms developing over time and gradually getting worse. Heart failure can also be left- and right-sided, systolic (with reduced ejection fraction) and diastolic (with preserved ejection fraction). NT-proBNP is used to diagnose or aid diagnosis of both acute and chronic heart failure.

### EXAMPLES

### General Methods and Materials

### Study Participants

Study participants included healthy volunteers and routine Royal Liverpool Hospital heart failure clinic outpatients with a diagnosed heart failure. All study participants were 18+ years of age and provided informed consent before donating clinical samples for this study. Participant exclusion criteria were the following: oral pathology including gingival disease, oral or oesophageal malignancy, Sjogren's syndrome, or any significant mucosal or oro-oesophageal abnormality, or refusal of consent.

### Sample Collection

Three types of clinical samples were obtained from each study participant: whole blood sample, whole saliva sample and OMT. Whole blood, OMT and whole saliva samples were collected in this order from each study participant.

Whole blood samples were collected by venipuncture into blood collection tubes e.g. vacutainers, CAT Serum separation tubes (VACUETTE®). The whole blood samples were immediately sent to Royal Liverpool Hospital lab for NT-proBNP immunoassay test. Serum was obtained by allowing the whole blood sample to clot at room temperature for between 30 and 60 minutes. The clotted blood was then removed by centrifugation at 2000×g for 15 minutes using a refrigerated centrifuge. The remaining aqueous layer (supernatant) is serum. Concentrations of NT-proBNP in the serum samples were then analysed using a biochemical NT-proBNP detection test (Roche Cobas 8000 e602/601 module (electrochemiluminescence)).

OMT samples were collected using Oracol® (Malvern Medical Developments Ltd) device. Study participants were asked to put the device between the lower gum and cheek at the left side for 5 minutes. After 5 minutes participants were asked to carry out a gentle rotation movement along the gum towards the right side with the device. Then the participants were asked to put the same device between the upper gum and cheek at the left side for 5 minutes. After 5 minutes participants were asked to carry out a gentle rotation movement along the gum towards the right side with the device and then place the Oracol device into 15 mL collection tube. Samples were transported to the lab on ice and then centrifuged at 3,000×g for 20 minutes at 4° C. The aqueous layer was put in the new 2 mL centrifuge protein LoBind tubes and centrifuged further at 10,000×g for 20 minutes at 4° C. The supernatant was collected in protein LoBind tubes and frozen at −80° C.

Whole saliva samples were collected in Saliva Collection Aid (Salimetrics LLC) via passive drool method. Unstimulated passive drool method was used to collect whole saliva instead of a stimulated swab collection, to mitigate dilution and interference effects that may be caused by stimulated saliva. Study participants were asked to tilt the head forward and let saliva drool into Saliva Collection Aid device for 5 minutes. Samples were transported to the lab on ice and then centrifuged at 17,000×g for 30 minutes at 4 C. The aqueous layer was collected in protein LoBind tubes and stored at −80 C.

Whilst Saliva Collection Aids and Oracol® devices were found to yield sufficient sample volumes for immunoassay analysis (see below) for the purpose of the examples provided herein, those skilled in the art will appreciate that devices other than Saliva Collection Aids and Oracol® may be used in accordance with the invention which are also eminently suitable for collecting whole saliva or OMT samples respectively.

### Immunoassay

NT-proBNP concentrations in blood, whole saliva and OMT were assessed by standard enzyme-linked immunosorbent assay (ELISA) techniques using human NT-proBNP antibodies. Human NT-proBNP ELISA Kit (ab263877, Abcam) was used to determine NT-proBNP concentrations in the samples. Whole saliva and OMT samples were thawed at ambient temperature before analysis. Wash buffer, antibody cocktail and standards were made up as per manufacturer instructions. Antibody cocktail consisted of primary Capture NT-proBNP antibody and secondary Detection NT-proBNP antibody. Assay standard concentrations were the following: 0 pg/mL, 21.9 pg/mL, 43.8 pg/mL, 87.5 pg/mL, 175 pg/mL, 350 pg/mL, 700 pg/mL, 1,400 pg/mL. 50 μL of standards and samples per well were loaded on 96-well plate in duplicates, followed by the addition of 50 uL of antibody cocktail to each well. The plate was sealed and incubated for 1 hour at room temperature as per manufacturer instructions. The plate was then washed three times with a wash buffer followed by the addition of 100 uL of TMB Development Solution to each well and incubation in the dark for 15 minutes. 100 uL of Stop Solution was then added to each well and the plate was read by VersaMax Microplate Reader at 450 nm as an endpoint reading. Quantification of NT-proBNP was performed using GraphPad Prism 6 software. Average absorbance value for the blank control (zero) standards was calculated and subtracted from all other standard absorbance values. Standard curve was created by plotting the average blank control subtracted absorbance value for each standard concentration (y-axis) against NT-proBNP concentration (x-axis) of the standard. Third order polynomial equation was used to interpolate the values of samples from the standard curve.

### Statistical Analysis

Data was interpolated using third order polynomial equation and Spearman's rank coefficient was used to test the correlation between whole saliva and OMT NT-proBNP concentrations vs serum NT-proBNP concentrations. Statistical analysis was performed using GraphPad Prism 6 software. D'Agostino & Pearson omnibus and Shapiro-Wilk normality tests were performed to test normal distribution of the data. Spearman's rank coefficient was calculated to determine a correlation between whole saliva NT-proBNP concentrations and serum NT-proBNP concentrations, and OMT NT-proBNP concentrations and serum NT-proBNP concentrations. One-tailed P value analysis was used. Receiver operating characteristic (ROC) curve analysis was used to analyse the diagnostic ability of a classifier system (e.g. a positive or a negative test result, also referred to herein as a ‘rule out test’). Receiver-operating characteristic (ROC) curve and ROC area under the curve (AUC) analyses were carried out to investigate the diagnostic applicability. Receiver-operating characteristic (ROC) curve and ROC area under the curve (AUC) analyses were carried out using GraphPad Prism 6 software. Optimal diagnostic cut-off level was calculated with highest sensitivity. Diagnostic applicability was expressed as AUC, cut-off level, sensitivity, specificity, negative predictive value (NPV) and positive predictive value (PPV). The Mann-Whitney test was used to compare whole saliva, OMT and serum NT-proBNP concentrations between healthy participants and heart failure patients cohorts.

### Example 1

### Blood, Whole Saliva and OMT Sample Collection

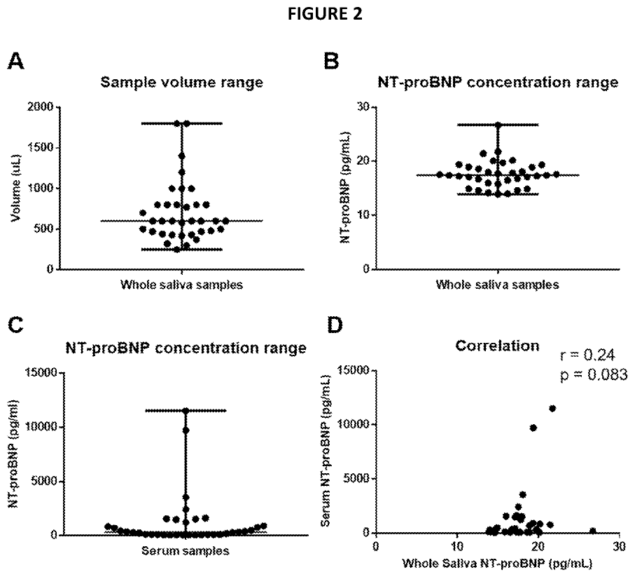

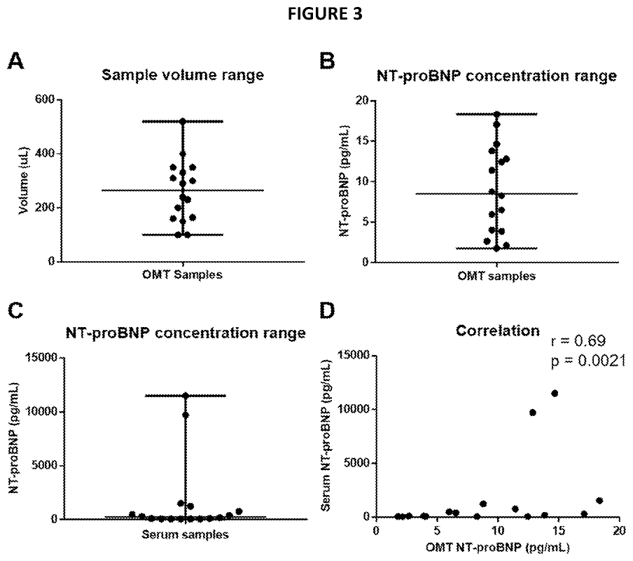

Blood, whole saliva, and OMT samples were obtained from each study participant. Blood samples were collected first, immediately followed by collection of OMT, immediately followed by collection of whole saliva. The number of study participants was 35. 14 study participants were reference subjects (i.e. did not have heart failure). 21 study participants had been diagnosed with heart failure. Blood samples were obtained as described above. Whole saliva samples were obtained using the passive drool method as described above. Whole saliva sample volumes ranged from 250 uL to 1800 uL, with a median sample volume equal to 600 uL (FIG. 2A, Table 1). OMT samples were successfully obtained from 16 study participants, using the collection methods as described above. Of these 16 OMT samples, 7 were obtained from reference subjects and 9 were obtained from study participants having heart failure. OMT sample volumes ranged from 100 uL to 520 uL, with a median volume of 265 uL (FIG. 3A, Table 1). Whole saliva samples were successfully obtained for 15/16 of the study participants from which OMT samples were successfully obtained.

TABLE 1
Volumes of collected whole saliva and volumes of collected OMT

| | | Sample Volume (μL) |
|---|---|---|
| Whole Saliva | Minimum | 250 |
| | Median | 600 |
| | Maximum | 1800 |
| OMT | Minimum | 100 |
| | Median | 265 |
| | Maximum | 520 |

### Example 2

### Concentration of NT-proBNP

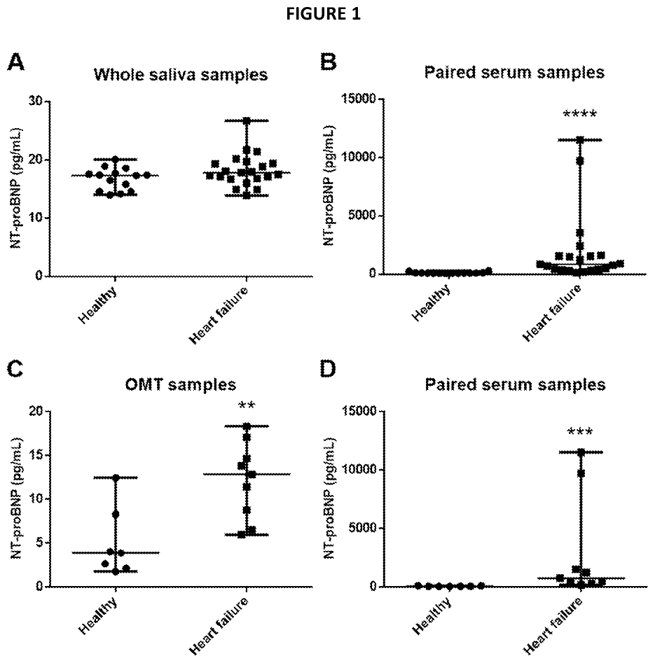

NT-proBNP concentration in blood samples, whole saliva and OMT samples were performed by an enzyme-linked immunosorbent assay as described above. NT-proBNP concentrations of whole saliva samples in healthy participants (n=14) ranged from 14.01 pg/mL to 20.09 pg/mL, with a median NT-proBNP concentration equal to 17.37 pg/mL (FIG. 1A, Table 2). NT-proBNP concentrations of whole saliva samples in heart failure patients (n=21) ranged from 13.9 pg/mL to 26.7 pg/mL, with a median NT-proBNP concentration equal to 17.83 pg/mL (FIG. 1A, Table 2). There was no significant difference between NT-proBNP concentrations of whole saliva samples in healthy participants and heart failure patients (p=0.1332). NT-proBNP concentrations of paired serum samples in healthy participants ranged from 59 pg/mL to 292 pg/mL, with a median NT-proBNP concentration of 87.5 pg/mL (FIG. 1B, Table 2). NT-proBNP concentrations of paired serum samples in heart failure patients ranged from 131 pg/mL to 11520 pg/mL, with a median NT-proBNP concentration of 844 pg/mL (FIG. 1B, Table 2). There was a strong significant difference between NT-proBNP concentrations of paired serum samples in healthy participants and heart failure patients (p<0.0001).

NT-proBNP concentrations of OMT samples in healthy participants (n=7) ranged from 1.77 pg/mL to 12.46 pg/mL, with a median NT-proBNP concentration equal to 3.89 pg/mL (FIG. 10, Table 2). NT-proBNP concentrations of OMT samples in heart failure patients (n=9) ranged from 5.98 pg/mL to 18.34 pg/mL, with a median NT-proBNP concentration equal to 12.85 pg/mL (FIG. 10, Table 2). There was a strong significant difference between NT-proBNP concentrations of OMT samples in healthy participants and heart failure patients (p=0.0052). NT-proBNP concentrations of paired serum samples in healthy participants ranged from 59 pg/mL to 122 pg/mL, with a median NT-proBNP concentration of 72 pg/mL (FIG. 10, Table 2). NT-proBNP concentrations of paired serum samples in heart failure patients ranged from 195 pg/mL to 11520 pg/mL, with a median NT-proBNP concentration of 768 pg/mL (FIG. 10, Table 2). There was a strong significant difference between NT-proBNP concentrations of paired serum samples in healthy participants and heart failure patients (p=0.0002).

TABLE 2
NT-proBNP concentrations in whole saliva, OMT and paired serum
samples in healthy participants and heart failure patients.

| | | | NT-proBNP |
|---|---|---|---|
| | | | concentration |
| Sample type | Cohort | | (pg/mL) |
| Whole Saliva | Healthy | Minimum | 14.01 |
| | participants | Median | 17.37 |
| | (n = 14) | Maximum | 20.09 |
| | Heart failure | Minimum | 13.90 |
| | patients | Median | 17.83 |
| | (n = 21) | Maximum | 26.70 |
| Paired serum | Healthy | Minimum | 59.00 |
| (for | participants | Median | 87.50 |
| Whole Saliva | (n = 14) | Maximum | 292.0 |
| sample type) | Heart failure | Minimum | 131.0 |
| | patients | Median | 844.0 |
| | (n = 21) | Maximum | 11520 |
| OMT | Healthy | Minimum | 1.77 |
| | participants | Median | 3.89 |
| | (n = 7) | Maximum | 12.46 |
| | Heart failure | Minimum | 5.98 |
| | patients | Median | 12.85 |
| | (n = 9) | Maximum | 18.34 |
| Paired serum | Healthy | Minimum | 59.00 |
| (for | participants | Median | 72.00 |
| OMT sample | (n = 7) | Maximum | 122.0 |
| type) | Heart failure | Minimum | 195.0 |
| | patients | Median | 768.0 |
| | (n = 9) | Maximum | 11520 |

For the purposes of correlation analysis healthy participants' and heart failure patients' NT-proBNP concentrations were pooled together in whole saliva cohort, OMT cohort and their paired serum cohorts.

Whole saliva sample NT-proBNP concentrations ranged from 13.9 pg/mL to 26.7 pg/mL, with a median NT-proBNP concentration equal to 17.42 pg/mL (FIG. 2B, Table 3). Serum NT-proBNP concentrations (for the whole saliva cohort n=35 participants) ranged from 59 pg/mL to 11520 pg/mL, with a median NT-proBNP concentration of 313 pg/mL (FIG. 2C, Table 3).

OMT sample concentration values ranged from 1.77 pg/mL to 18.34 pg/mL, with a median concentration of 8.54 pg/mL (FIG. 3B, Table 3). Serum NT-proBNP concentrations (for the OMT cohort n=16 participants) ranged from 59 pg/mL to 11520 pg/mL, with a median NT-proBNP concentration of 254 pg/mL (FIG. 3C, Table 3).

TABLE 3
NT-proBNP concentrations in whole saliva,
OMT and paired serum samples.

| | | | NT-proBNP |
|---|---|---|---|
| | | | concentration |
| Cohort | | | (pg/mL) |
| Whole Saliva | Whole | Minimum | 13.9 |
| Cohort | Saliva | Median | 17.42 |
| (n = 35 | | Maximum | 26.7 |
| participants) | Serum | Minimum | 59 |
| | | Median | 313 |
| | | Maximum | 11520 |
| OMT Cohort | OMT | Minimum | 1.77 |
| (n = 16 | | Median | 8.54 |
| participants) | | Maximum | 18.34 |
| | Serum | Minimum | 59 |
| | | Median | 254 |
| | | Maximum | 11520 |

These data show, for the first time, that NT-proBNP is detectable specifically in OMT. We have also demonstrated that NT-proBNP is detectable in human whole saliva. We identified a weak positive correlation between whole saliva NT-proBNP concentration and serum NT-proBNP concentration, which was not statistically significant. The lack of correlation between whole saliva NT-proBNP concentrations and serum NT-proBNP concentrations reconfirms previous findings.

### Example 3

### Statistical Analysis

The NT-proBNP concentrations for the whole-saliva cohort (n=35 participants) and the OMT cohort (n=16 participants) was found to have a non-normal distribution (Table 4).

TABLE 4
Serum and whole saliva NT-proBNP concentration data; and NT-proBNP and
OMT concentration data do not pass normality tests. Test A - D'Agostino
& Pearson omnibus normality test. Test B - Shapiro-Wilk normality test.

| | Whole Saliva Cohort | | OMT Cohort | |
|---|---|---|---|---|
| | (n = 35) | | (n = 16) | |
| | NT-proBNP (pg/mL) | | NT-proBNP (pg/mL) | |
| | Serum | Whole Saliva | Serum | OMT |
| Test A | | | | |
| K2 | 49.72 | 14.73 | 20.75 | 2.605 |
| P Value | <0.0001 | 0.0006 | <0.0001 | 0.2718 |
| Passed | No | No | No | Yes |
| normality test | | | | |
| (alpha = 0.05) | | | | |
| P value | **** | *** | **** | Not significant |
| summary | | | | |
| Test B | | | | |
| W | 0.48 | 0.9146 | 0.5047 | 0.9387 |
| P Value | <0.0001 | 0.01 | <0.0001 | 0.3331 |
| Pass | No | No | No | Yes |
| normality test | | | | |
| (alpha = 0.05) | | | | |
| P value | **** | * | **** | Not significant |
| summary | | | | |
| Sum | 42090 | 618.5 | 26786 | 144.7 |

In view of the non-normal data distribution, to evaluate correlation between whole saliva NT-proBNP concentration and serum NT-proBNP concentrations, and between OMT NT-proBNP concentration and serum NT-proBNP concentrations, Spearman's rank correlation tests were performed. No correlation between whole saliva NT-proBNP concentrations and serum NT-proBNP concentrations was observed (Spearman's rank correlation coefficient r=0.24, p=0.083; FIG. 2D). A strong positive correlation between OMT NT-proBNP concentrations and serum NT-proBNP concentrations was observed (Spearman's rank correlation coefficient r=0.69, p=0.0021; FIG. 3D).

### Example 4

### Diagnosis Applicability

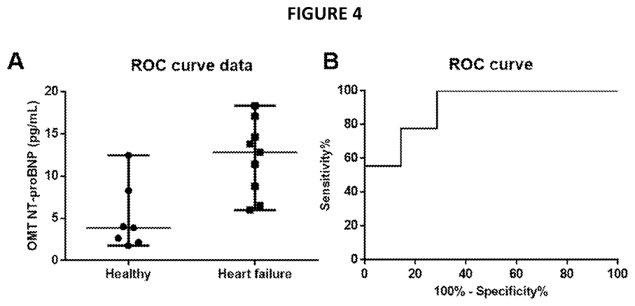

To demonstrate the clinical utility of OMT in diagnosing heart failure we compared OMT NT-proBNP concentrations of healthy participants and OMT NT-proBNP concentrations of heart failure patients. Receiver operating characteristic (ROC) analysis provided an OMT NT-proBNP cut-off value of 5.01 pg/mL, yielding 100% sensitivity and 71% specificity with area under the curve (AUC) of 0.9 (FIG. 4A-B; Table 5). Negative and positive predictive values were 100% and 81.6%, respectively (Table 5).

TABLE 5

| | | | | | | Neg- | Posi- |
|---|---|---|---|---|---|---|---|
| | | | | | | ative | tive |
| Area | | 95% CI | Cut- | | | pre- | pre- |
| Under | | (confi- | off | | | dictive | dictive |
| Curve | P | dence | (pg/ | Sensi- | Specif- | value | value |
| (AUC) | value | interval) | mL) | tivity | icity | (NPV) | (PPV) |
| 0.9 | 0.007 | 0.75-1.06 | 5.01 | 100% | 71% | 100% | 81.6% |

ROC AUC analysis of OMT NT-proBNP.

These data demonstrate the diagnostic applicability of OMT NT-proBNP in detecting heart failure in a subject. As a strong rule out test must always reside on the side of high sensitivity, the inventors have demonstrated OMT NT-proBNP cut-off of 5.01 pg/mL provides a diagnostic test that is up to 100% sensitive with negative predictive value up to 100%.

1. A method of aiding diagnosis of a disease or condition in a subject, wherein the method comprises comparing a level of at least one natriuretic peptide detected in a biological sample obtained from the subject with a reference level of the at least one natriuretic peptide, wherein the biological sample is an oral mucosal transudate (OMT) sample obtained from one or more mucous membranes inside the mouth of the subject.

2. A method according to claim 1, wherein the one or more mucous membranes inside the mouth of the subject are located at the gingiva, buccal mucosa, alveolar mucosa, labial mucosa, junctional epithelium, or combinations thereof.

3. A method according to claim 1 or claim 2, wherein the biological sample is an OMT sample obtained from the surface of the one or more mucous membranes inside the mouth of the subject.

4. A method according to claim 3, wherein the OMT sample obtained from the subject is obtained from the surface of the gingiva in the mouth of the subject.

5. A method according to claim 4, wherein the OMT sample is obtained from the surfaces of the gingiva and the alveolar mucosa in the mouth of the subject.

6. A method according to claim 4 or claim 5, wherein the OMT sample is obtained from the surfaces of the gingiva, alveolar mucosa and labial mucosa in the mouth of the subject.

7. A method according to any one of claims 4-6, wherein the OMT is obtained from the surfaces of the gingiva, alveolar mucosa and buccal mucosa in the mouth of the subject.

8. A method according to any one of claims 1 to 7, wherein the OMT sample is obtained from a particular location of one or more mucous membranes inside the mouth of the subject.

9. A method according to claim 8, wherein the OMT sample is obtained from a particular location of the gingiva, optionally wherein the particular location is one or more of the gingival sulcus, the marginal gingiva, free gingiva, interdental gingiva, gingival groove, mucogingival junction and the attached gingiva, optionally still wherein the particular location is from the gingival sulcus.

10. A method according to any one of claims 1-9, wherein the OMT sample comprises gingival crevicular fluid, optionally wherein the OMT sample is gingival crevicular fluid.

11. A method according to any one of claims 1 to 10, wherein the at least one natriuretic peptide is selected from BNP or NT-proBNP, optionally wherein the at least one natriuretic peptide is NT-proBNP.

12. A method according any one of claims 1 to 11, wherein the concentration of the at least one natriuretic peptide in the OMT sample obtained from the subject is from 0.5 pg/mL to 200 pg/mL, optionally from 1 pg/mL to 50 pg/mL, optionally still from 1 pg/mL to 40 pg/mL.

13. A method according to any one of claims 1-12, wherein the disease or condition is a heart condition, optionally wherein the heart condition is heart failure.

14. A method according to any one of claims 1-13, wherein the method comprises determining whether the level of the at least one natriuretic peptide is increased compared to the reference level, wherein an increased level is indicative of the disease or condition.

15. A method according to any one of claims 1 to 14, wherein the reference level is a reference concentration of the at least one natriuretic peptide in an OMT sample.

16. A method according to claim 15, wherein the reference concentration is 10 pg/mL or less, optionally 8 pg/mL or less, optionally 5 pg/mL or less, and optionally still 4 pg/mL or less.

17. A method of aiding the assessment of a disease or condition in a subject identified as having or suspected of having the disease or condition, the method comprising comparing the level of at least one natriuretic peptide detected in a first biological sample obtained from the subject before treatment for the disease or condition, with the level of at least one natriuretic peptide detected in a second biological sample obtained from the subject during or after treatment for the disease or condition, wherein the first and second biological samples are independently OMT samples obtained from one or more mucous membranes inside the mouth of the subject.

18. A method according to claim 17, wherein the method comprises:
detecting the level of the at least one natriuretic peptide in the first biological sample obtained from the subject before treatment for the disease or condition; and
detecting the level of the at least one natriuretic peptide in the second biological sample obtained from the subject during treatment for the disease or condition, or after treatment for the disease or condition has ended.

19. A method according to claim 18 wherein:
an increase in, or maintenance of, the level of the at least one natriuretic peptide from the first biological sample to the second biological sample is indicative of an ineffective treatment for the disease or condition; and
a decrease in the level of the at least one natriuretic peptide from the first biological sample to the second biological sample is indicative of an effective treatment for the disease or condition.

20. A method according to any one of claims 17-19, wherein the disease or condition is a heart condition, optionally wherein the heart condition is heart failure

21. A method according to any of the preceding claims, wherein the level of the at least one natriuretic peptide in the biological sample is determined using a mass spectrometry technique, an optical technique or a ligand-binding technique.

22. A method according to claim 21, wherein the level of the at least one natriuretic peptide is determined using a ligand-binding technique, wherein the ligand-binding technique is an immunogenic technique.

23. A kit for performing a method according to any of the preceding claims, wherein the kit comprises one or more reagents for use in a method of detecting the at least one natriuretic peptide, optionally wherein the at least one natriuretic peptide is NT-proBNP.

24. Oral mucosal transudate (OMT) obtained from a subject for use in a method of diagnosing a disease or condition in a subject, optionally wherein the disease or condition is a heart condition, optionally wherein the heart condition is heart failure.
